(19) United States
(12) Patent Application Publication
(10) Pub. No.: US 2015/0087975 A1
(43) Pub. Date: Mar. 26, 2015

(54) ELASTOGRAPHY-BASED ASSESSMENT OF CRYOABLATION

(71) Applicants: Septimiu Salcudean, Vancouver (CA); Caitlin Schneider, Vancouver (CA); Diego Dall'Alba, Santorso (IT); Robert Rohling, Vancouver (CA); Christopher Nguan, Vancouver (CA)

(72) Inventors: Septimiu Salcudean, Vancouver (CA); Caitlin Schneider, Vancouver (CA); Diego Dall'Alba, Santorso (IT); Robert Rohling, Vancouver (CA); Christopher Nguan, Vancouver (CA)

(73) Assignee: UNIVERSITY OF BRITISH COLUMBIA, Vancouver (CA)

(21) Appl. No.: 14/035,843

(22) Filed: Sep. 24, 2013

(51) Int. Cl. A61B 8/08; A61B 18/02

(52) U.S. Cl. CPC A61B 8/0858; A61B 18/02; A61B 2018/00577
USPC 600/438; 606/20

(57) ABSTRACT

A method of monitoring the cryoablation of a target volume of tissue with ultrasound elastography, the method comprising acquiring a first elastography image encompassing said target volume of tissue, performing at least one cycle of freezing and thawing of tissue encompassed in said target volume, acquiring a second elastography image encompassing said target volume, and comparing said first and said second elastography images over said target volume. The elastography provides either relative or quantitative measurements of tissue elasticity. The elastography maps of tissue elasticity, before and after cryoablation of one region, can guide the cryoablation of another region. The use of elastography provided feedback to the operator to achieve effective treatment with cryoblation over a planned target.

### FIELD OF THE INVENTION

The present invention relates to tissue cryoablation and, in particular, to a method of assessing the result of cryoablation for quality assurance and treatment re-planning.

### BACKGROUND OF THE INVENTION

The basis of medical imaging is the measurement of a property of tissue that varies with tissue composition. Medical images are formed by displaying intensities as a function of these properties measured at various locations in the body. Mechanical properties of tissue are important indicators of disease potential. Indeed, palpation techniques are commonly used by medical doctors to determine the potential for disease, for example, stiffer tissue regions that feel harder can indicate the presence of cancer. This is the basis for a number of clinical examinations such as the digital rectal examination for prostate cancer. A change in the mechanical properties of tissue can also be an indicator of the success or failure of therapy.

Elastography is a medical imaging modality that aims to depict elasticity, a mechanical property of tissue. Elasticity is also referred to as stiffness, or the inverse compliance. For this imaging technique, a mechanical excitation is applied in the proximity of the tissue of interest (e.g., the prostate) and the resulting deformation is measured. Typically the resulting deformation is measured with ultrasound (ultrasound elastography or USE) or Magnetic Resonance Imaging (magnetic resonance elastography or MRE). The deformation is post-processed to extract information such as viscoelastic properties (e.g., shear modulus and viscosity). The deformation or tissue strain, or alternatively, the intrinsic mechanical properties of tissue are then displayed as a map of stiffness (or other meaningful mechanical property) of the imaged object.

Clinical uses of elastography were first demonstrated in the field of ultrasound as described in U.S. Pat. No. 5,107,837 by Ophir et. al. entitled “Method and Apparatus for Measurement and Imaging of Tissue Compressibility and Compliance.” Shortly afterwards elastography was introduced in the field of magnetic resonance imaging (MRI) by Ehman and Muthupillai as described in U.S. Pat. No. 5,825,186 entitled “Method for Producing Stiffness-Weighted MR Images” and U.S. Pat. No. 5,977,770 by Ehman entitled “MR Imaging of Synchronous Spin Motion and Strain Waves.” In the following years elastography was shown to be of clinical value for the detection and staging of hepatic (liver) fibrosis by Sinkus et. al. “Liver fibrosis: non-invasive assessment with MR elastography” in the journal NMR in Biomedicine 2006, pages 173-179, and Ehman et. al. “Assessment of Hepatic Fibrosis With Magnetic Resonance Elastography” in the Journal of Clinical Gastroenterology and Hepatology, volume 5, Issue 10, Oct. 2007, pages 1207-1213. Elastography imaging of the breast has been successfully demonstrated and published by Sinkus et. al. in “Viscoelastic shear properties of in vivo breast lesions measured by MR elastography” in the Journal of Magnetic Resonance Imaging volume 23, 2005, pages 159-165. Elastography of the brain was also published by Papazoglou and Braun et. al. in “Three-dimensional analysis of shear wave propagation observed by in vivo magnetic resonance elastography of the brain” in Acta Biomaterialia, volume 3, 2007, pages 127-137. More recently, elastography of the lung was demonstrated by Ehman et. al. In U.S. Pat. No. 2006/0264736 entitled “Imaging Elastic Properties of the Lung with Magnetic Resonance Elastography.” MRE of the prostate ex-vivo was demonstrated first by Dresner, Rossman and Ehman, published in the Proceedings of the International Society for Magnetic Resonance in Medicine entitled “MR Elastography of the Prostate” in 1999. MRE of the prostate in-vivo was demonstrated by Sinkus et. al. and published in “In-Vivo Prostate Elastography”, Proceedings of International Society of Magnetic Resonance in Medicine, volume 11, page 586, 2003. Prostate elastography is described in U.S. Pat. No. 5952828, 2010/0005892, 7,034,534 and the publications referred to above and also by Kemper, Sinkus et. al. “MR Elastography of the Prostate: Initial In-vivo Application.” published in Fortschritte auf dem Gebiete der Rontgenstrahlen and der Nuklearmedizin (Advances in the area of X-ray and Nuclear Medicine), volume 176, pages 1094-1099, 2004. An alternative approach to prostate elastography uses excitation applied through the rectum or the urethra as described in U.S Pat. No. 2009/0209847 and 2010/0045289 and the following publication Plewes et. al. “In Vivo MR Elastography of the Prostate Gland Using a Transurethral Actuator” Magnetic Resonance in Medicine, volume 62, 2009, pages 665-671. Alternatively, the mechanical excitation can be applied by a needle that penetrates the skin as described in U.S. Pat. No. 2008/0255444.

Alternatively the mechanical excitation can be applied through the perineum as described in U.S. patent application Ser. No. 13/104,081 by Salcudean et al.

Percutaneous ablation procedures are minimally invasive surgical techniques with very encouraging medium term outcomes. In this procedure, an elongated instrument, called ablation probe, is inserted through the skin of the patient in order to reach the target zone. The ablation probe is able to deliver high energy by means of radio-frequency or microwave frequency electromagnetic fields, high energy focused ultrasound, lasers, etc. Cryotherapy involves local freezing and thawing of tissue which causes both direct injury to cells and secondary injury due to the inflammatory response of the body.

In general, cryoablation provides some advantages compared to other percutaneous ablation approaches: it is relatively easy to use with multiple probes at the same time, ice provides a natural anesthetic effect, and the region of treatment, usually referred to as an ice ball or freezing zone, presents homogeneous characteristics. This translates, at least with respect to kidney tumors, to better mid-term outcomes, reduced recurrence and easier follow-up protocols (Heuer, Roman, Inderbir S Gill, Giorgio Guazzoni, Ziya Kirkali, Michael Marberger, Jerome P Richie, and Jean J M C H de la Rosette (2010), “A critical analysis of the actual role of minimally invasive surgery and active surveillance for kidney cancer.” European Urology, 57, pp. 223-232.)

During percutaneous ablation procedures, the physician carrying out the intervention has no direct visual feedback so a medical imaging modality is required for guidance. The progress of thermal ablation can be successfully monitored with both ultrasound and magnetic resonance imaging. For example, in U.S. Pat. No. 7,792,566B2, a system is presented for thermal ablation using high intensity ultrasound with the temperature monitored by a volumetric MR image. In U.S. Pat. No. 5,657,760, ultrasound Doppler imaging is used to monitor the extent of tissue damage induced by various thermal modalities. In U.S. Pat. No. 7,846,096, the difference in ultrasound raw echo image sequences acquired at intervals of a few seconds or less during ablation treatment are used to determine a difference image which is filtered and used to generate an indication of the effect of a discrete ultrasound medical treatment. Many of the ablation monitoring technique involve the use of temperature monitoring, e.g. (T. Varghese J A Zagzebski et al, “Ultrasound monitoring of temperature change during radiofrequency ablation: Preliminary in-vivo results”, Ultrasound in Med and Biol, 28(3), pp321-329, 2002), and (V. Rieke, A. M Kinsey, A B Ross, “Referenceless MR thermometry for monitoring thermal ablation in the prostate”, IEEE Transactions on Medical Imaging, 26(6), pp 813-821, 2007). Alternatively, instead of using temperature monitoring, the tissue coagulation induced by the high temperature in thermal ablation can be monitored directly by MRI (Chen L, Bouley D, Yuh E, Butts DAHK. Study of focused ultrasound tissue damage using MRI and histology. J Magn Reson Imaging 1999; 10:146-153.)

MR elastography monitoring of thermal tissue ablation has been demonstrated in (T. Wu, J. P Felmlee, J F Greenleaf, S J. Riedere, R L Ehman, “Assessment of thermal tissue ablation with MR elastography”, Magnetic Resonance in Medicine, 45(1), 80-87, January 2001). In this work, it is shown that associated with thermal ablation, there is an irreversible change in tissue elastic properties of tissue that can be measured with MRE.

The use of ultrasound elastography images to provide visualization in two and three dimensions of radio-frequency ablation has been described in U.S. Pat. No. 7,166,075, in which the ablation radiofrequency probe is used to apply a tissue compression. In US Patent Application 2010/0256530 A1, ultrasound elastography has been proposed as a way to measure ablation from radio frequency or microwave frequency electrical energy. The method comprises a vibrating ablating electrode, an imager to image shear waves introduced by the vibrating electrode in tissue, and a computer program to compute the change in shear wave velocity through the ablated region as the region is being heated in order to output data on the size of the ablation region.

It is well known that real-time monitoring of cryoablation with conventional ultrasound is not possible because during the freezing cycle of the cryoablation process, an ice ball is found that overlaps with the tumour region to be ablated. This is discussed, for example, in (U Lindner, J Trachtenberg, N Lawrentschuk, “Focal therapy in prostate cancer: modalities, findings and future considerations, Nature Reviews Urology 7, 562-571, October 2010”). Alternatively, B-Mode ultrasound images are used in ultrasound guided cryoablation to visualize the frozen zone after the complete thawing, but these images do not provide good contrast of the region (Onik, G M, G Reyes, J K Cohen, and B Porterfield , “Ultrasound characteristics of renal cryosurgery.” Urology, 42, 212-215, 1993). In this work, a comparison is reported based on the maximum diameter measured along the sagittal direction in the ultrasound B-mode image and in histological analysis, and errors of up to 5 mm are reported. In (Janzen, Nicolette K, Kent T Perry, Ken-Ryu Han, Blaine Kristo, Steven Raman, Jonathan W Said, Arie S Belldegrun, and Peter G Schulam, “The effects of intentional cryoablation and radio frequency ablation of renal tissue involving the collecting system in a porcine model.” The Journal of Urology, 173, 1368-74, 2005), results of cryoablation lesion localization based on the area difference between B-mode ultrasound and pathological analysis are reported. This work confirms that the measurements obtained from ultrasound are not reliable, with underestimation up to 18.5% and overestimation up to 260%.

Therefore, the boundaries of the cryoablated areas cannot be monitored with standard B-mode ultrasound as the ablation proceeds. In real-time monitoring, ultrasound imaging breaks down because of strong reflections and shadows from the ice ball. For assessment of a complete cryoablation freeze-thaw cycle, standard B-mode ultrasound is not accurate enough. Therefore new methods are required for the effective monitoring or assessment of cryotherapy.

### BRIEF DESCRIPTION OF THE DRAWINGS

FIG. 1 is a side (sagittal) view of a patient undergoing cryoablation of the prostate under ultrasound guidance.

FIG. 2. is a detailed view of a possible ultrasound transducer used to guide the cryoablation procedure and the ultrasound imaging planes it can produce.

FIG. 3 is an illustrative view of the imaging volume that the ultrasound transducer in FIG. 2 can produce, the contour of the prostate being imaged, a possible cryoablation needle with a schematic cryoablation volume it can produce, a schematic of a planning target volume that may contain cancer, and of two other cryoablation volumes, obtained with the cryoablation needle at different locations, in order to cover the planning target volume.

FIG. 4A is an illustrative view of a sagittal cross section of the imaging volume with the outline of a planned target volume overlayed on top. FIG. 4B is an illustrative view of a transverse cross section of the imaging volume with the outline of a planned target volume overlayed on top.

FIG. 5 is an illustrative view of a sagittal plane cross-section through a prostate elastography image, with the image being acquired first prior to cryoablation (left) and after cryoablation (center), with the difference between images being shown on the right. Overlays on these images are cross-sections of the boundary of the planned target volume and a stiffer tumor within it.

FIG. 6 is an illustrative view of the temperature profile that was followed in a freeze-thaw cycle applied to freshly excised pig kidneys.

FIG. 7 shows the mean elasticity and measurement range of the freshly excised pig kidneys prior to ablation and after thawing.

FIG. 8 shows the required positioning of a cryoablation needle in order to cover an area that did not show a significant decrease in elasticity.

### DETAILED DESCRIPTION OF EMBODIMENTS OF THE INVENTION

We propose to use the elasticity of tissue, measured with ultrasound-based elastography, to determine whether a region of tissue has been properly cryoablated. Our description will be with reference to a cryoablation system for the prostate, but it is understood that this reference to the prostate is in no way limiting and that applications to other organs will be similar and easily adapted to by someone skilled in the art.

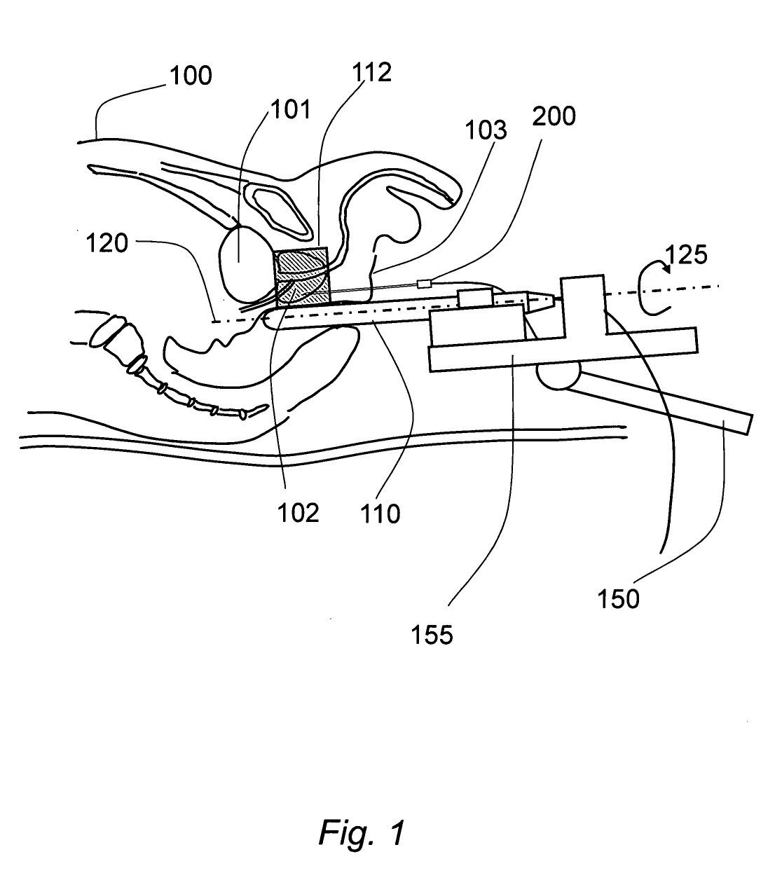

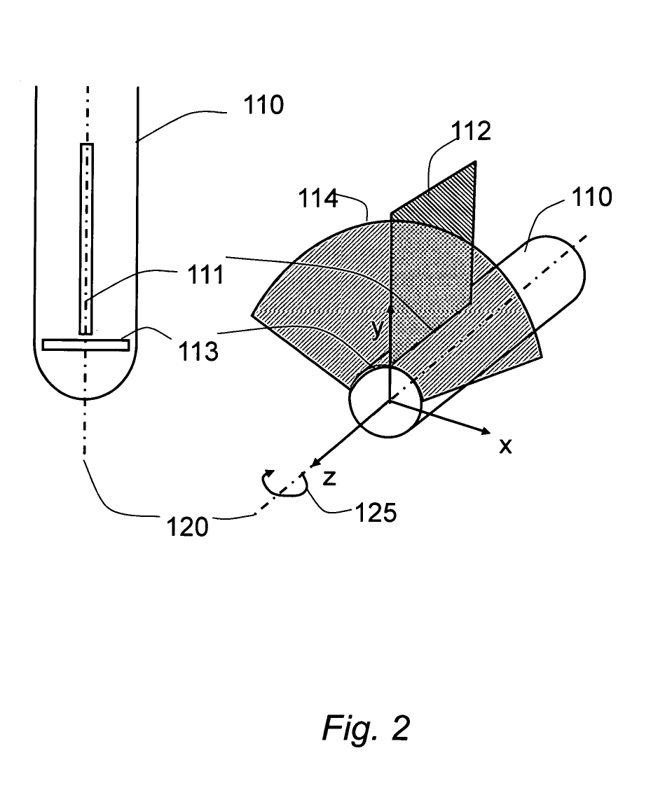

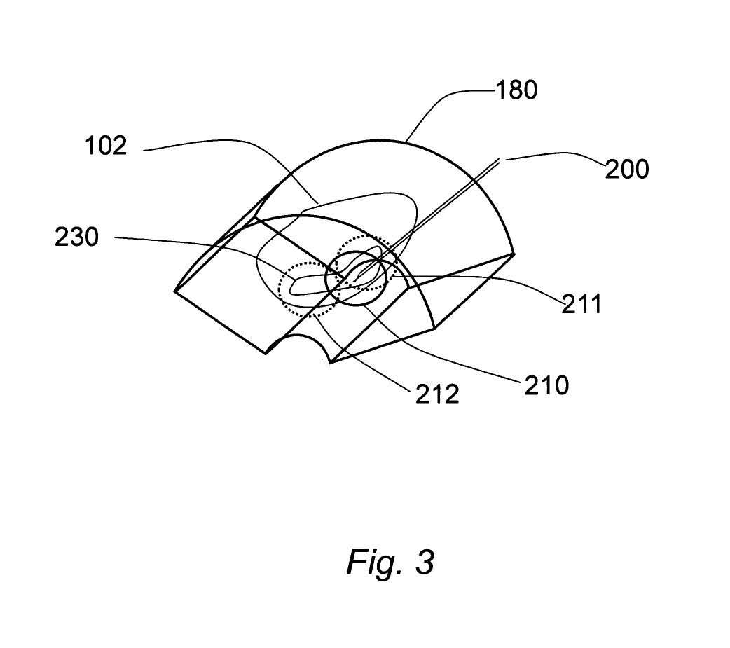

Aspects of the invention are described with reference to FIGS. 1 through 5 and FIG. 8 as applied to prostate ablation. For a cryoablation procedure, the patient 100 is being imaged by an ultrasound transducer 110 that images the prostate 102 as well as some of the periprostatic region including part of the bladder 101. The transducer is connected to a brachytherapy stabilizer 150 and stepper 155, as exemplified by the CIVCO MicroTouch stabilizer with the EXII Brachytherapy Stepper. The transducer, shown in FIG. 2, is a brachytherapy transducer having a convex side-firing imaging array 113 producing a sector planar image 114 and a linear sagittal array 111 producing a rectangular image 112. We will call the image 112 sagittal, even though it may be at an angle with respect to the sagittal plane of the patient when the transducer is rotated about its own axis 120. Both the translation of the transducer 110 along its main longitudinal axis 120 and the rotation of the transducer 125 around the main longitudinal axis 120 are encoded, as done in the commercial CIVCO EXII Brachytherapy Stepper, and read by a computer to allow for the generation of 3D images contained in a cylindrical sector volume 180 as shown in FIG. 3, as known to practitioners of 3D ultrasound. Examples of acquisition of 3D images with such a system are reported, for example, in (S. S. Mandavi, M. Moradi, X. Wen, W. J. Morris, and S. E. Salcudean, “Evaluation of visualization of the prostate gland in vibro-elastography images”, medical image Analysis, Vol 15, Issue 4, August 2011, pp. 589-600).

Within the sector volume view of the ultrasound transducer, an ablation volume 210 corresponding to the cryoablation needle 200, such as the 17-gauge cryoablation needle manufactured by Galil Medical, is shown. The cryoablation needle is shown to be inserted through the perineum 103 of the patient 100. The cryoablation needle may be guided to the targeted area by a brachytherapy template (as available for the CIVCO ExII Brachythreapy stepper) calibrated with the ultrasound volume 180, by an embedded electromagnetic sensor on the needle and in the ultrasound transducer, as available on many ultrasound machines and in particular on the Ultrasonix GPS option (Ultrasonix Medical Corporation, Richmond, BC), or by a robotic needle guide calibrated to the ultrasound transducer, as described, for example, in (S. E. Salcudean, D. Prananta, W. J. Morris and I. Spadinger, “A robotic needle guide for prostate brachytherapy, IEEE Intl. Conf. on Robotics and Automation, pp. 2975-2981, 19-23 May 2008) or references therein.

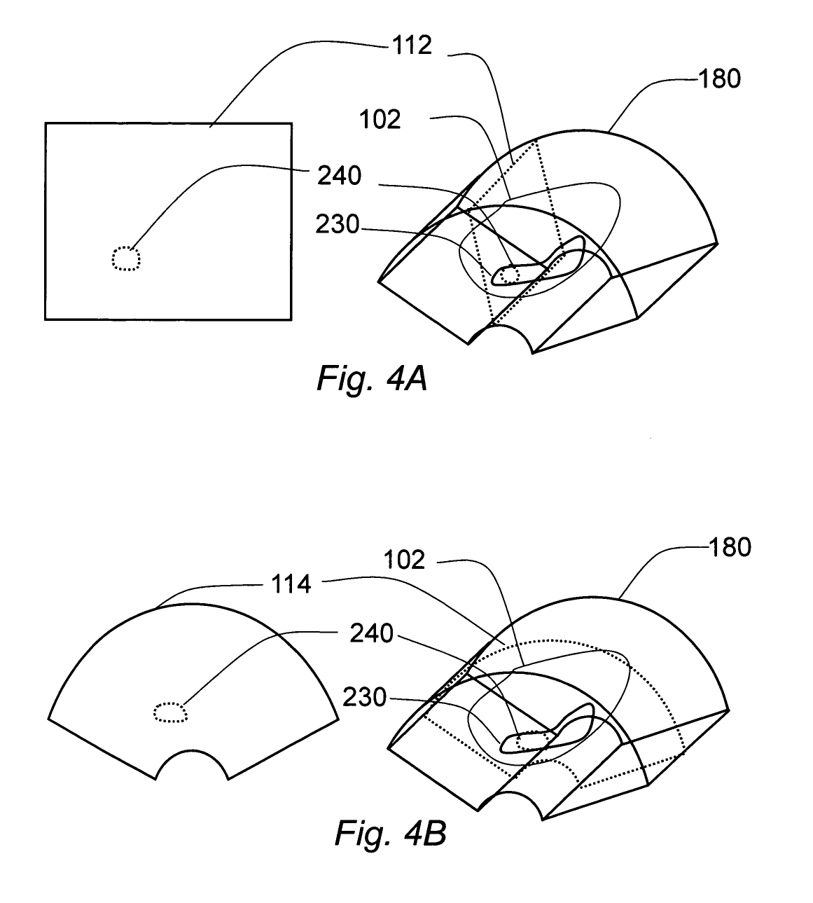

The planned ablation volume is illustrated as a set of three ablation volumes 210, 211, 212 at three different positions of the cryoablation needle 200 covering the planning target volume to be ablated 230. The cryoablation treatment consists of the possibly repeated application of freeze-thaw cycles with the cryoablation needle at the positions corresponding to the ablation volumes 210, 211,212. The goal of the procedure is to cover the planning target volume 230 that may be known to contain cancer from a previously taken biopsy or may be suspicious of being cancerous due to a prostate imaging study. The prostate imaging study may include B-mode ultrasound, Doppler ultrasound, elastography, radiofrequency ultrasound signal analysis in terms of features (E. J. Feleppa, A. Kalisz, J B Sokil-Melgar, F. L. Lizzi et al, “Typing of prostate tissue by utraosinic ultrasonic spectrum analysis, IEEE. Trans on Ultrasonic, Ferroelectronics and Frequency Control, vol 43, issue 4, pp. 609-619, July 1996) or radiofrequency time-series analysis (M. Moradi, P. Abolmaesumi and P. Mousavi, “Tissue typing using ultrasound RF time series: experiments with animal tissue samples”, Medical Physics, vol 37, issue 8, 2010). The prostate imaging study may include another imaging modality that is registered to the ultrasound imaging volume 180 by various algorithms known in the art (Farheen Taquee, O. Goksel, S S, Mandavi et al, “Deformable. Prostate Registration from MR and TRUS Images Using Surface Error Driven FEM models”, Proc. SPIE, 2012), an references therein. The planning target volume 230 may be stored in the computer or the ultrasound machine and may be shown as a rendered overlay on a rendered 3D view of the imaged volume 180. Preferably, as shown in FIG. 4a, as the sagittal imaging plane 112 crosses the planning target volume 230, the pixels 240 on the planning target volume boundary may be highlighted, or, alternatively in FIG. 4b, as the transverse imaging plane 114 crosses the planning target volume 230, pixels 240 on the planning target volume boundary may be highlighted.

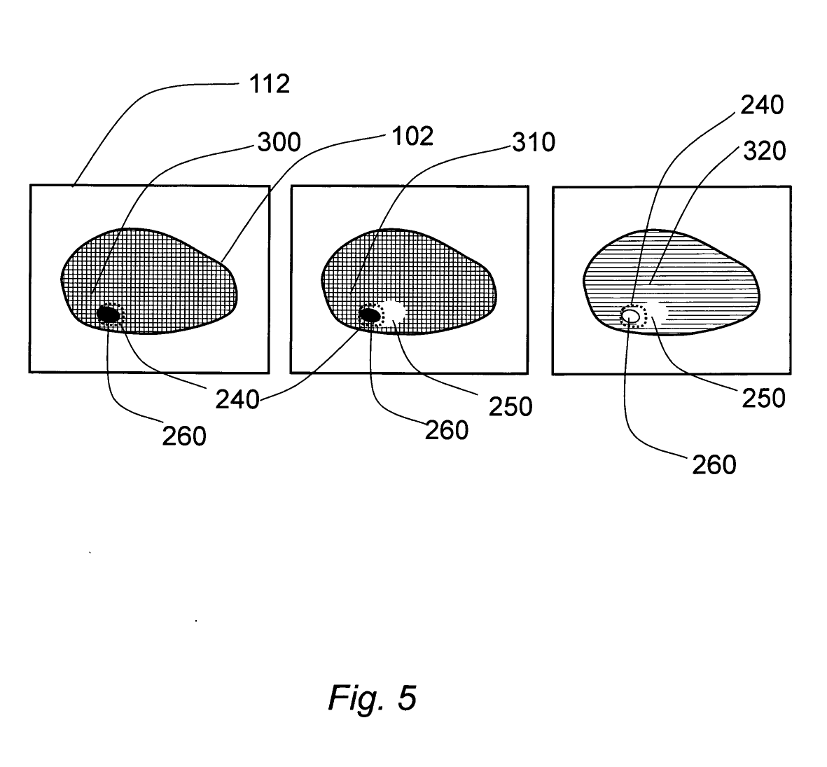

In one aspect of the invention, a first elastography image of the imaging volume 180 is taken prior to the treatment commencing, and a second elastography image of the image volume is taken after the ablation (freezing and thawing) is performed. The two quantitative images may be displayed as 3D images side by side with the planning target volume 230 as a transparent rendered overlay as known in the state of the art on each of the images. Preferably, as shown in FIG. 5, the boundary 240 of the planning target volume 230 is simultaneously displayed as an overlay on the first elastography image cross section 300 defined by the sagittal plane 112, and as an overlay on the second elastography image cross section 310 defined by the same sagittal plane 112. Alternatively, the boundary 240 of the planning target volume 230 is displayed as a 3D overlay on the difference between the first and second elastography image. Alternatively, the boundary 240 of the planning target volume 230 can be displayed as an overlay on the cross section 320 defined by the sagittal plane 112 through the difference between the first and the second elastography image.

The cross sections of the elastography images 300 and 310 show the outline of the prostate, which is usually stiffer than the surrounding tissue, and also a lesion 260, shown darker, or stiffer. The cross-section 250 of the ablated volume that is crossed by the sagittal imaging plane 112 through the imaging volume 180 is shown in FIG. 5 as a white region 250. It is shown both in the post-cryo-ablation elastography image cross-section 310, and in the difference between the pre and post cryo-ablation elastography images 320.

Alternatively, the boundary 250 of the planning target volume 230 is shown as it is crossed by the transverse plane 114 in each of the two elastography images, with these images being shown simultaneously side by side. Alternatively, the boundary 250 of the planning target volume 230 is shown as it is crossed by the transverse plane 114 on the difference between the first and the second elastography images. The images would be similar to those shown in FIG. 5 and are not shown here.

It is obvious that in the above description, the imaged volume 180 does not have to encompass the full 3D volume that is capable of being produced by the ultrasound imaging device. The same approach can be clearly used with a thin elastography image volume or with one or multiple elastography imaging planes.

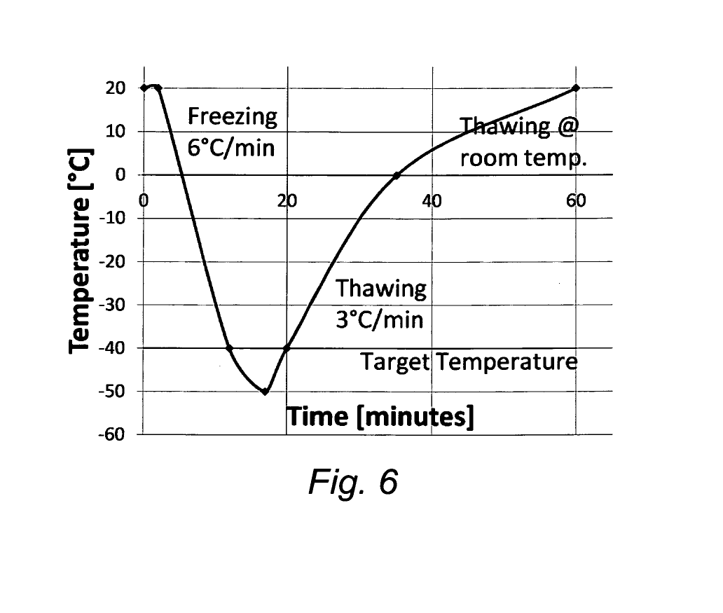

The elastography image will show a change as a result of the ablation procedure. In particular, the inventors have demonstrated that as a result of the mechanical stresses caused by ice crystal formation on the tissue microscopic structure, the tissue macroscopic elastic properties are also affected. In particular, the inventors have obtained quantitative elastography images of pig kidneys before cryoablation and after cryoblation. Freezing was obtained by immersion in a bath of dry-ice and acetone with a low conductivity coupling container protecting the sample against cryo-shock. The freezing speed was 6° C./minute and thawing speed was 3° C./minute. Each cycle took approximately 35 minutes plus 25 minutes to ensure that the temperatures before and after the experiment were the same. The temperature profile is reported in FIG. 6.

The quantitative elastography method employed has been described in PCT Patent Application PCT/CA2012/000779, Baghani et al, filed on Aug. 17, 2012, the entirety of which is herein incorporated by reference. A summary of the method is presented in in (Ali Baghani et al “Real-Time Quantitative Elasticity Imaging of Deep Tissue Using Free-Hand Conventional Ultrasound”, in The 15^{th} Intl Conf. on Medical Image Computing and Computer Assisted Intervention, 1-5 Oct. 2012). It consists of applying a multi-frequency excitation to tissue, measuring the tissue motion over a volume using ultrasound (A. Bahgani, S. E. Salcudean and R. Rohling, A High Frame Rate US System for the Study of Tissue Motions. IEEE Trans. UFFC 2010), and solving an inverse problem or finding the local spatial frequency estimate of the wave motion and then the shear wave speed and therefore the shear modulus (A. Manduca et al., Local wave-length estimation for MRE. IEEE Int. Conf. on Image Proc. 1996).

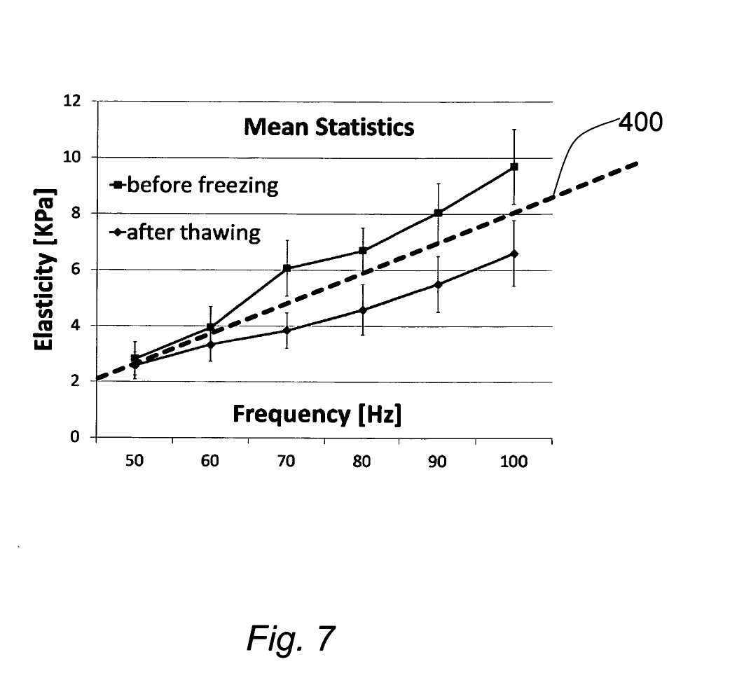

Excitation was provided by an external device (LDS Mod. V203, B&K, Denmark), controlled between 50 Hz and 100 Hz. The data were acquired with an ultrasound imaging system (Sonix Touch, Ultrasonix, Canada) with a 3D mechanical probe (4DL14-5/38, at 5 MHz). A region of interest in the quantitative elastography data was manually selected within the volume and the mean value and the standard deviation of elasticity are reported in FIG. 7. As can be seen, a significant difference between tissue elasticity before and after the freezing is present, in the frequency range from 70 HZ to 100 Hz, where the curves can be separated even when taking into account for the measurement range displayed at each frequency. The significance of the test was also confirmed by a Tukey's Honestly Significant Difference test (a <0.05).

In order to obtain elastography images by using the method described in PCT Patent Application PCT/CA2012/000779 by Baghani et al, filed on Aug. 17, 2012, in a clinical environment, an excitation must be generated that propagates through tissue. This can be generated by motion of the ultrasound transducer, as described in U.S. patent application Ser. No. 12/240,895 by Salcudean et al. The excitation can be generated by applying an exciter on the body, e.g. on the perineum, as described in U.S. patent application Ser. No. 13/104,081 by Salcudean et al.

Alternatively, other elastography techniques can be used to obtain the tissue elastography images acquired before and after the cryoablation procedure. These include Shear Wave imaging on the Aixplorer ultrasound machine (Supersonic Imagine), acoustic radiation impulse force imaging, vibro-elastography (U.S. Pat. No. 7,731,661, Salcudean, Rohling and Turgay), and many other methods that have been published or patented on the topic. Elastography can be a strain image, shear wave image, shear wave velocity image, Young's modulus image, viscosity image or any other image that depicts a variation in the mechanical properties of tissue.

Preferably the elastography methods used will be quantitative, in that they will provide not just a strain image, which provides a relative elasticity measure that depends on the operator, but also quantitative.

The difference between the elasticity image obtained before the cryoablation procedure and after the cryoablation procedure provides us with a quality assurance test based on elastography: a region of tissue will be considered to be adequately ablated if its measured quantitative elasticity at a frequency or over a frequency range falls below the line 400 (FIG. 7) found to separate the measured elasticities found before freezing and after thawing. In general, the exact elasticity decrease will be dependent on the tissue type but the same principle will apply.

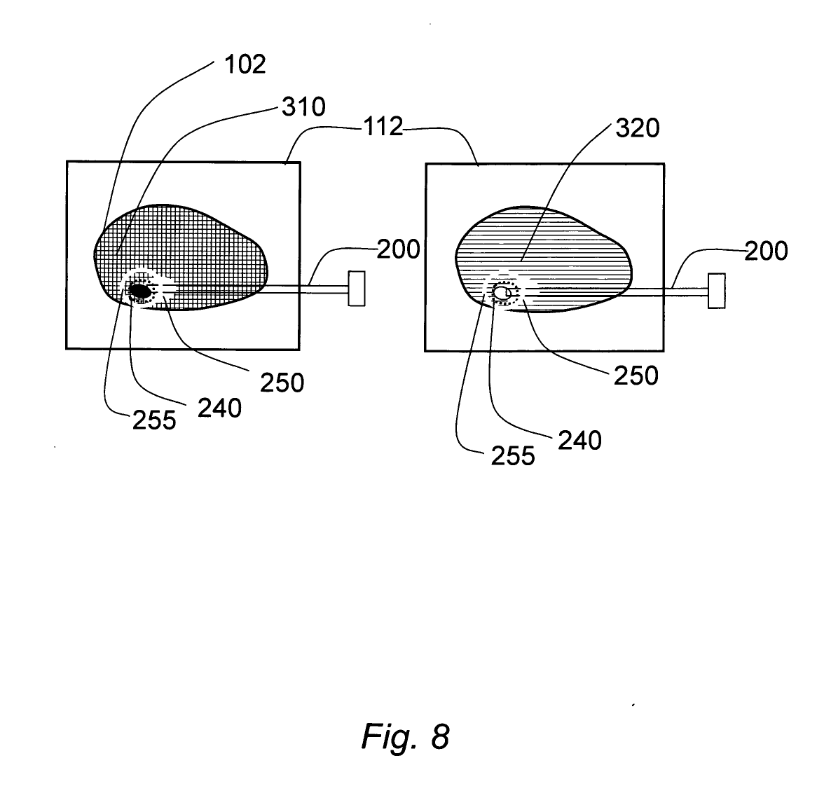

With reference to FIG. 8, it can be seen that if a comparison of the elastography images from before and after ablation show an ablation region 250 that does not fully encompass the planning target volume 230, then a new ablation volume can be defined by adjusting the position of the cryoablation needle in order to generate an adjusted ablation volume. Such adjustment can be carried out manually, by using multiple images through various sagittal planes 112 or multiple images through various transverse planes 114, or both, and finding the largest discrepancy between the planned ablation region 240 and the actual ablation region 250, and using this largest discrepancy to determine the direction in which the cryoablation needle needs to be adjusted. Assuming, for example, that FIG. 5 shows such largest discrepancy, the cryoablation region needs to have another cryoablation region developed by a cryoablation needle 200 inserted further into the prostate along the plane 112, as shown in FIG. 8, towards left or the base of the prostate, in order for the new cryoblation region 255 to fully cover the target 240. This process can be repeated until the image to be cryoablated fully encloses the planned target volume.

Alternatively, a computer planning system may be used to make changes to an ablation plan as a result of taking elastography images before and after cryoablation. In particular, such a planning system can use the regions from the elastography images that do not show a decrease in elasticity due to cryoablation, in order to design a minimum number of ablation regions that would completely encompass the planned target volume.

1. A method of monitoring the cryoablation of a target volume of tissue with ultrasound elastography, the method comprising acquiring a first elastography image encompassing said target volume of tissue, performing at least one cycle of freezing and thawing of tissue encompassed in said target volume, acquiring a second elastography image encompassing said target volume, and comparing said first and said second elastography images over said target volume.

2. A method as in claim 1, wherein said first and said second elastography images are quantitative.

3. A method as in claim 1, further comprising comparing said first and said second elastography images over a volume outside said target volume.

4. A method as in claim 1, wherein said comparing said first and said second elastography images over said target volume comprises comparisons over corresponding imaging planes.

5. A method for adjusting the cryoablation of a target volume of tissue with ultrasound elastography, the method comprising acquiring a first elastography image of said target volume of tissue, performing at least one cycle of freezing and thawing of tissue encompassed in said target volume, acquiring a second elastography image of the target volume, comparing said first and said second elastography images to select a second target region within said target volume and adjusting the location of the cryoablation to target said second target region.
